(19) United States
(12) Patent Application Publication
(10) Pub. No.: US 2007/0048725 A1
(43) Pub. Date: Mar. 1, 2007

(54) Machine perfusion of tissue grafts for transplantation

(76) Inventor: Ben O'Mar Arrington, East Stroudsburg, PA (US)

Correspondence Address: BLANK ROME LLP, 600 NEW HAMPSHIRE AVENUE, N.W., WASHINGTON, DC, 20037, US

(21) Appl. No.: 11/509,017

(22) Filed: Aug. 24, 2006

Related U.S. Application Data
(60) Provisional application No. 60/710,881, filed on Aug. 25, 2005.

(51) Int. Cl. A01N 1/02; A61M 31/00

(52) U.S. Cl. 435/1.1; 435/2; 604/500

(57) ABSTRACT

Methods and apparatuses for exsanguination or replacement of blood in a tissue are presented. These methods and apparatuses may be used to preserve and prepare an organ or other tissue for transplantation while increasing the likelihood of a successful procedure. Improved methods are also provided for the splitting of organs and other tissues.

### REFERENCE TO RELATED APPLICATION

The present application claims the benefit of U.S. Provisional Patent Application No. 60/710,881 filed Aug. 25, 2005, whose disclosure is hereby incorporated by reference in its entirety into the present application.

### FIELD OF THE INVENTION

The invention relates to the field of organ and biological tissue preservation. In particular, the invention relates to machine perfusion for the preservation of organs and biological tissues for implant and/or transplant.

### BACKGROUND

Organ transplantation is the only treatment option for people with end stage organ disorders. While organ transplants continue to become more common and successful, lack of usable donor organs prevents a large number of people from having a transplant. For example, one of the donor organs that is most difficult to obtain is the liver. According to the Scientific Registry of Organ Transplant, at the end of 2004, more than 17,000 people in the United States were on the waiting list to receive a liver transplant. By contrast, in the same year, fewer than 6,000 liver transplants were performed, and approximately 1,800 people on the waiting list died without a transplant. It is of primary concern in the field to develop better methods for preserving and preparing donor organs for transplant to maximize the number of organs that are available for use.

Improved processes for preservation and preparation of donor organs will allow a broader range of available organs to be made amenable for transplantation. Increasing the time for which an organ can be preserved is important, as it allows for organ sharing among transplant centers, careful preoperative preparation of the recipient, time for preliminary donor culture results to become available, and time for vascular repairs of the organ prior to implantation. Further, a method is needed that not only increases the storage time of an organ, but actually provides a way to make the organ more amenable to transplant. There are many donor organs that are considered marginal, usually because their donors are elderly or have certain medical conditions. Marginal organs often show delayed graft function which can lead to the failure of a transplant.

Cold storage (CS) is the standard preservation technique for most organ transplantation procedures. CS preservation is a straightforward procedure that involves flushing the organ with cold preservation solution followed by submersion and storage of the organ in cold preservation solution. While CS preservation is adequate for many organs, many transplant centers have resorted to more aggressive use of marginal livers in response to the growing waiting list (see Rocha et al. Transplant Proc. 2004 May; 36(4):914-5 for example).

Preservation injury is a major mechanism of graft disfunction, especially in marginal or injured grafts. The ischemia and reperfusion events that occur during preservation account for only part of this damage. For vascular organs, such as the liver and kidneys, damage from ongoing metabolism can also be acute. Most current organ preservation methods, such as CS, are designed to quickly cool the organ and keep it in a static state. Cooling the organ has the effect of slowing metabolism greatly, but does not stop it. Thus, there are still cellular substrates being consumed and metabolites being produced. For vascular organs, the process of metabolism can be especially damaging, as these organs naturally function in the presence of blood flow that provides the necessary substrates to and removes wastes products from the system.

There are several tests that can be done on the effluent of a harvested organ to predict its suitability for transplant. Tests for a specific enzymatic activity or concentration of a metabolite can help determine whether an organ has internal damage that might lead to poor graft function. During CS, there is no perfusate flowing through the organ, making it impossible to periodically monitor its condition. For marginal organs to be successfully utilized, it is desirable to be able to monitor the condition of the organ from the time it is removed from the donor until it is transplanted into the recipient. This way, the transplant team will know if an organ has a reasonable chance of displaying good graft function.

Another technique that has grown in popularity due to the great shortage of organs is organ splitting. Organ splitting involves separating the organ into two or more functioning parts and transplanting each part into a separate patient. This way, one donor organ can be made into two or more grafts. While organ splitting has shown great promise to provide for more patients in need of a transplant, current organ splitting techniques have several disadvantages which prevent their widespread use. Presently, organ splitting is performed either ex situ during CS in situ or during procurement of the organ.

Ex situ organ splitting performed during CS has several distinct disadvantages. As the organ needs to be manipulated, there is the risk of it being warmed by operating room lights and the surgeon's hands. As described above, warming of the organ increases the rate of the metabolic processes of the organ, causing damage to cells and tissue. Further, because there is no storage fluid flowing through the organ, there is no way to tell if its vessels are intact. Leaking vessels that are not repaired can cause blood loss and hamper graft function after transplant.

While in situ organ splitting avoids the problems associated with ex situ splitting, it is a complicated procedure requiring great manpower and expense. Because of its complexities, in situ organ splitting can add up to four hours to the organ procurement process. In situ organ splitting is often performed by the most experienced surgeon of the transplant unit, which often requires that the surgeon travel to a donor site far from the site where the transplant will be done. This leaves the transplant team without its most senior member for consultation. Also, because in situ splitting must be performed in the presence of low blood flow, other organs that could be procured from the donor may be damaged during the procedure.

Overall, improvements in preservation, pre-transplant assessment, ex vivo resuscitation and organ splitting have the potential to safely maximize utilization of the donor pool. Because the scarcity of quality organs is the major problem affecting the global efficiency of transplantation, the results of a better method for preparing organs for transplant will be felt almost immediately.

### SUMMARY OF THE INVENTION

From the above, it should be apparent that the need exists for an improved method of preserving organs for transplantation that allows for the use of a broader range of donor organs. An object of the present invention is to provide a method and apparatus for preserving a tissue from the time it is removed from a donor until it is transplanted into a recipient. More specifically, one object of the present invention is to provide a method and apparatus for preserving a tissue for transplantation by machine perfusion. Unlike CS, machine perfusion provides continuous circulation, delivers metabolic substrates, removes waste products, and improves microvascular integrity during preservation. Another benefit is that machine perfusion allows dynamic assessment of the graft quality during perfusion. It has also been shown that machine perfusion improves early graft function in kidney transplantation, especially for marginal organs.

A further object of the present invention is to provide a perfusion apparatus for exsanguination or replacement of blood in a tissue. The apparatus includes a compartment for holding the tissue to be treated and a pump to deliver aqueous medium to the tissue. The design of the pump is such that it causes the aqueous medium to flow through the apparatus and the tissue with a continuous, laminar, low shear, low turbulence flow. This type of flow greatly reduces the damage to the tissue that can be caused by perfusion.

A further object of the present invention is to provide an apparatus for exsanguination or replacement of blood in a tissue that is portable. A portable apparatus has particular utility in the preservation of donor organs, where the organs may need to be transported.

A further object of the present invention is to provide a method for the exsanguination or replacement of blood using the perfusion apparatuses provided by the present invention.

A further object of the present invention is to provide a method for ex vivo treatment of a tissue. During ex vivo treatment, a tissue may be perfused with an aqueous medium containing a therapeutic agent. This ex vivo treatment is not limited to treatment of tissue to undergo allograft transplants (transplantation into another), but also for autologous re-implant.

A further object of the present invention is to provide a method and apparatus for improved ex vivo splitting of a tissue, such as an organ. In this aspect, the tissue is split by standard methods while being machine perfused, allowing for more successful transplantation of the resulting grafts.

A still further object of the present invention is to provide a method and apparatus for a pharmacological treatment model of a tissue such as an organ. In this aspect, the invention is used to simulate an isolated tissue system, wherein blood, a blood replacement solution, or a perfusate is flowing through the tissue in a way that may be easily manipulated and controlled. In this type of system, the effects of therapeutic agents on the tissue may be easily monitored.

### BRIEF DESCRIPTION OF THE DRAWINGS

FIG. 1 is a schematic diagram of one embodiment of the apparatus of the invention for machine perfusion of human liver grafts;

FIG. 2 is a side view of a second embodiment of the invention in the form of a portable machine perfusion apparatus;

FIG. 3 is a schematic of a portable machine perfusion apparatus in accordance with the invention with one-piece construction wherein the temperature of the system is regulated by convection cooling and convection heating;

FIG. 4 is a schematic of a portable machine perfusion apparatus in accordance with the invention with one-piece construction wherein the temperature of the system is regulated by conduction cooling and convection heating;

FIG. 5 is a schematic of a portable machine perfusion apparatus in accordance with the invention with two-piece construction wherein the temperature of the system is regulated by conduction cooling and convection heating;

FIG. 6 is a cross-sectional side view of the portable machine perfusion apparatus shown in general in FIG. 2 and further diagrammed in FIG. 4;

FIG. 7 is an exploded cross-sectional view of the organ compartment of the portable machine perfusion apparatus shown in FIGS. 2 and 6 detailing the fluid circulation pattern;

FIG. 8 is a general cross-sectional side view of the organ compartment of the portable machine perfusion apparatus shown in FIGS. 2 and 6;

FIG. 9 is a flow diagram for the portable machine perfusion apparatus of the invention, wherein the electronics and transducers of the apparatus are disposable;

FIG. 10 is a flow diagram for the portable machine perfusion apparatus of the invention, wherein only the transducers of the apparatus are disposable;

FIG. 11 is a block diagram of the systems control of the portable machine perfusion apparatus of the invention, showing the input and output signals of the control computer;

FIG. 12 is a circuit diagram of the custom signal amplifier used in the perfusion apparatus of the invention; and

FIGS. 13A and 13B illustrate trends of post-transplant liver function tests in miniature swine, comparing machine preservation and cold storage. 13A shows the amount of aspartate transaminase activity present post-transplant. 13B Shows the concentration of Bilirubin present post-transplant.

### DETAILED DESCRIPTION OF THE INVENTION

The present invention is a method and apparatus for exsanguination or replacement of blood with aqueous treatment medium in body tissues such as vascular organs. An aspect of the invention is that the exsanguination or blood replacement is performed in a manner that leaves little damage to the organ or tissue and preserves all functional cells.

The method and apparatus of the present invention have varied uses, non-limiting examples of which are set forth in this specification. For the purposes of this specification, uses of the present invention for blood replacement, unless otherwise noted, are also uses of the present invention for exsanguination and the terms may be used interchangeably herein. Also, for the purposes of this specification, use of the present invention with a tissue or organ can mean use of the system with a single organ, a plurality of organs, or a body tissue or tissues, unless otherwise noted.

The present invention provides methods and apparatuses for exsanguination or replacement of blood with preservative or treatment medium in vascularized organs and vascular tissue while preserving all functional cells. In one embodiment, the invention is used for preserving an organ for transplant or other use using machine perfusion. Specific embodiments of the invention are set forth below using liver as an example donor organ. These examples should not be construed to limit the scope of the invention to use with donor livers. It should be apparent to one of skill in the art that other organs and tissues fall within the scope of the present invention, for example, hearts, kidneys, pancreases, and lungs.

Throughout this description, like elements are referred to by like numbers as shown in the drawings with increments of 100 between figures.

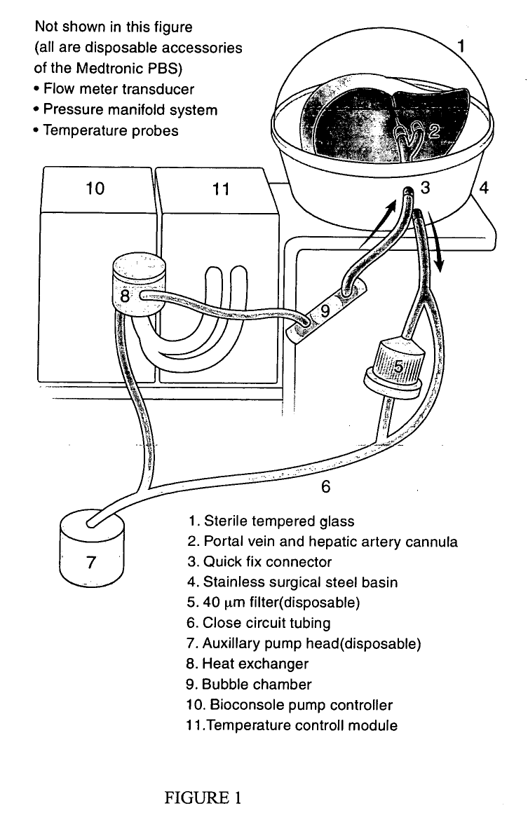

In one embodiment, the machine perfusion apparatus of the invention is a modification of the Medtronic Portable Bypass System® (PBS) sold by Medtronic, Inc., which is described in U.S. Pat. No. 5,823,986. This embodiment is as shown in the schematic of FIG. 1.

In this embodiment of the invention, the tissue graft is continuously perfused with cold machine preservation solution by the perfusion apparatus. The apparatus, unlike commercially available organ preservation devices, utilizes a centrifugal pump that can deliver a constant flow of preservation solution with laminar, low shear flow. Flow is adjusted using the pump controller 10, and the temperature is maintained by the blood temperature controller 11. The apparatus is set up and used as described in the Medtronic PBS manual for the apparatus as an extracorporeal bypass circuit, except that the connections will be to the stainless surgical steel basin 4 containing the liver graft rather than to the patient, and no oxygenation will be used.

During removal of the donor organ, the surgeon procures a segment of donor aorta from the diaphragm to the superior mesenteric artery en bloc with the liver graft. This segment of aorta does not interfere with procurement of any other organ. The proximal aorta will be cannulated by the surgeon using a reusable metal cannula attached to the PBS tubing. This technique of arterial cannulation using a segment of donor aorta is identical to the technique used for cannulation of the renal artery for machine perfusion of kidneys.

The surgeon then secures a second metal cannula to the superior mesenteric/portal vein junction. During procurement of the graft, excess length of superior mesenteric and portal vein will be obtained, which permits insertion of the cannula without injury to the main portal vein, which is used for vascular reconstruction during transplantation.

The liver graft is then placed in a closed surgical steel basin 4, which is covered with a sterile tempered glass lid, 1. The cannulae are connected to the circuit's bypass tubing via quick fix connectors 3. The apparatus has a centrifugal pump that delivers cold preservation solution to the graft via the aorta and mesenteric vein cannulas 2, and recirculates effluent solution collected from the hepatic veins. Effluent also forms a bath of preservation solution around the liver, topically cooling the surface of the graft. The PBS therefore accomplishes two functions in its interface with the graft: continuous delivery of substrates to the tissue and core cooling of the tissue via the console's built-in heat exchanger 8. The protocol of this specific embodiment does not utilize oxygenation nor is a membrane oxygenator a part of the circuit, but such elements could be present in other embodiments of the invention.

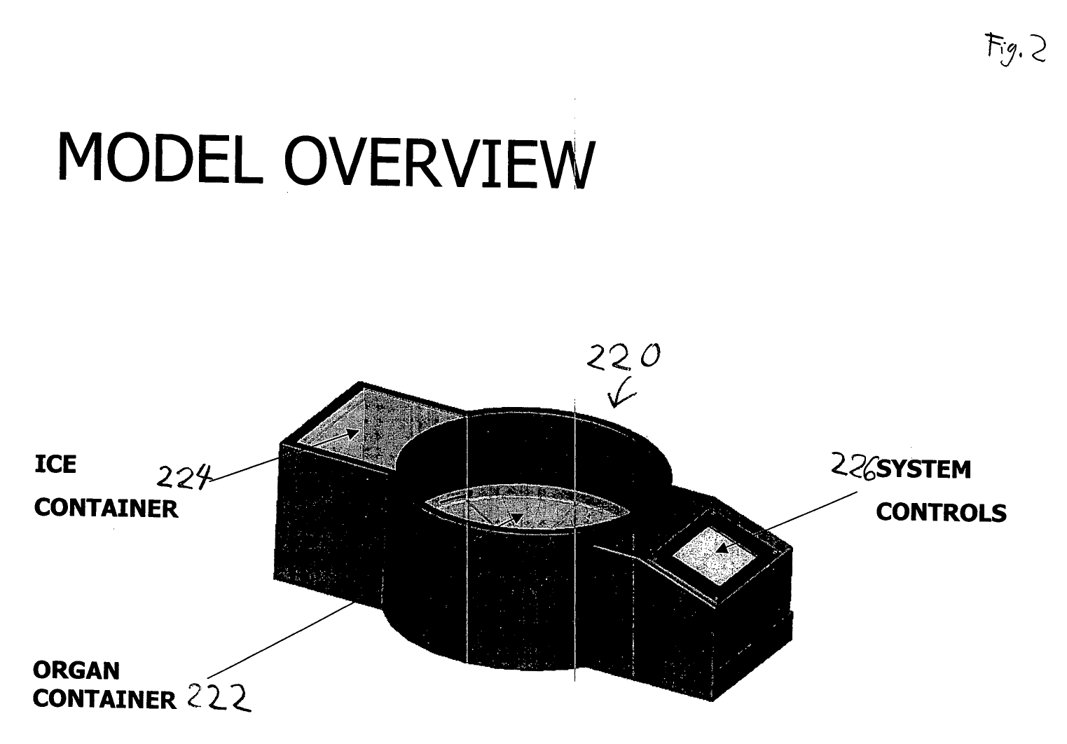

In a second embodiment, the machine perfusion apparatus is contained in a portable unit as shown schematically in FIG. 2. Generally, the portable machine perfusion apparatus 220 comprises a tissue container 222, a removable ice container for cooling 224, and system control computer 226, for controlling the pump speed and system temperature.

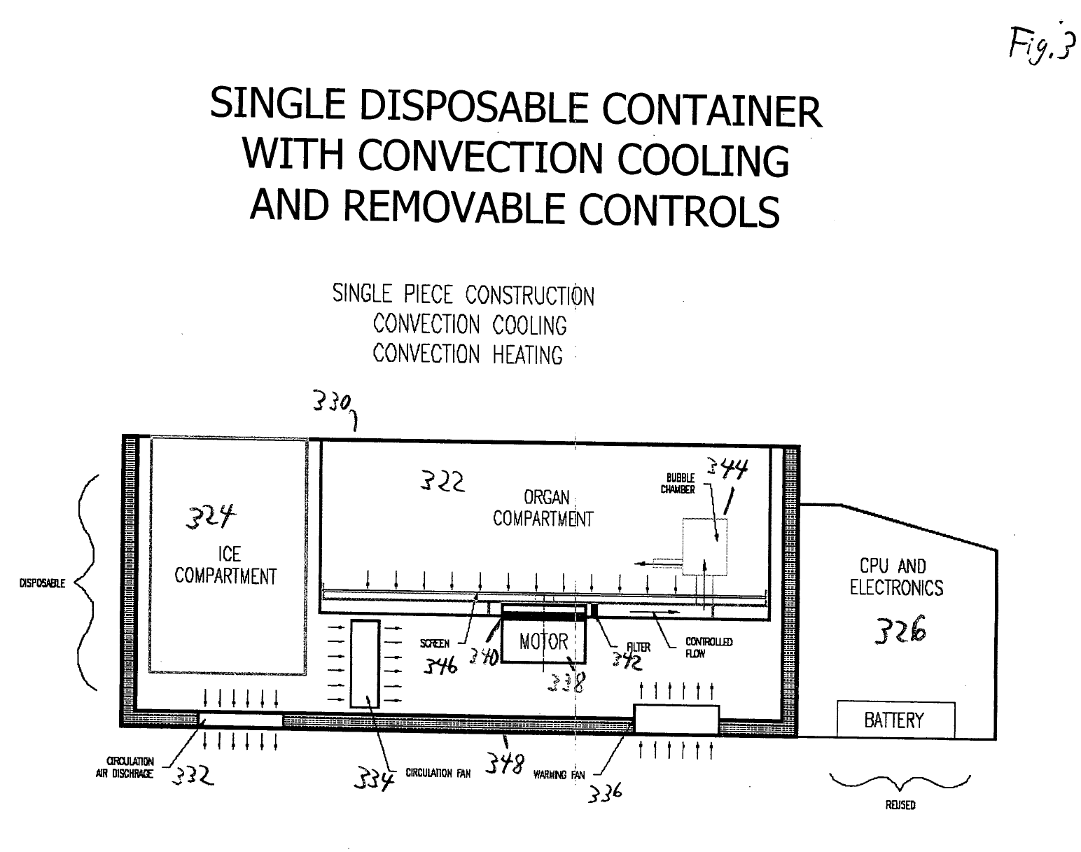

The temperature of the portable apparatus can be maintained by various methods. In one embodiment of the invention, the temperature of the apparatus is maintained by a convection method, as pictured in the schematic of FIG. 3. In the convection model 330, the temperature of the system is controlled by a discharge vent 332, a circulation fan 334 and a warming fan 336. Cold air surrounding the removable ice container 324 is either circulated by the circulating fan 334 or discharged by the air discharge vent 332. Warm air is brought into the system by the warming fan 336. The speed of the fans is regulated by the system control computer 326 to maintain the desired temperature of the tissue and the perfusion fluid. Perfusion fluid is circulated by a pump comprising a motor 338 and an impeller 340 controlled by the system control computer 326 which pumps the fluid in a controlled manner through a filter 342, a bubble chamber 344 and finally through the tubing that connects the bubble chamber with the cannulated tissue into the tissue. After passing through the tissue, effluent passes through a screen 346 in the floor of the tissue container 322 and into the pump inlet to be re-circulated through the system. The entire housing of the system, except for the system control computer 326, is encased in an insulated enclosure 348. In one embodiment of the invention all of the components except for the system control computer 326 are disposable, as denoted in FIG. 3.

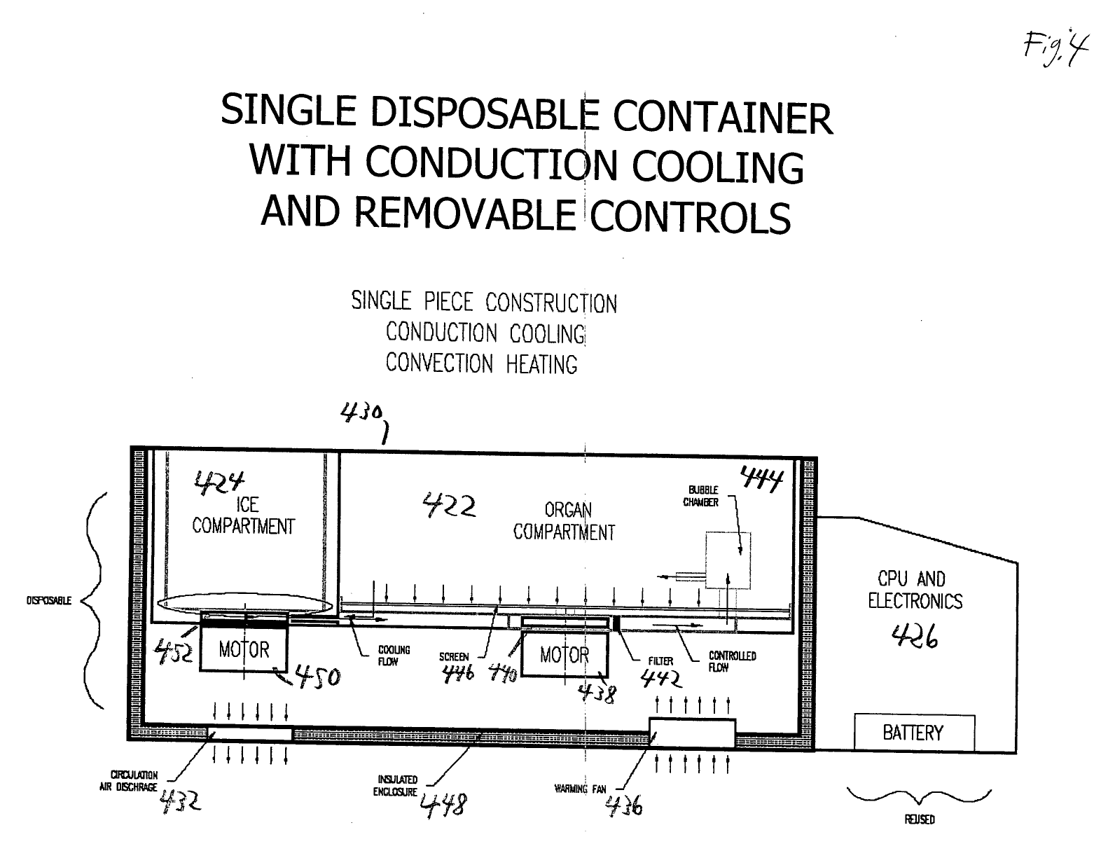

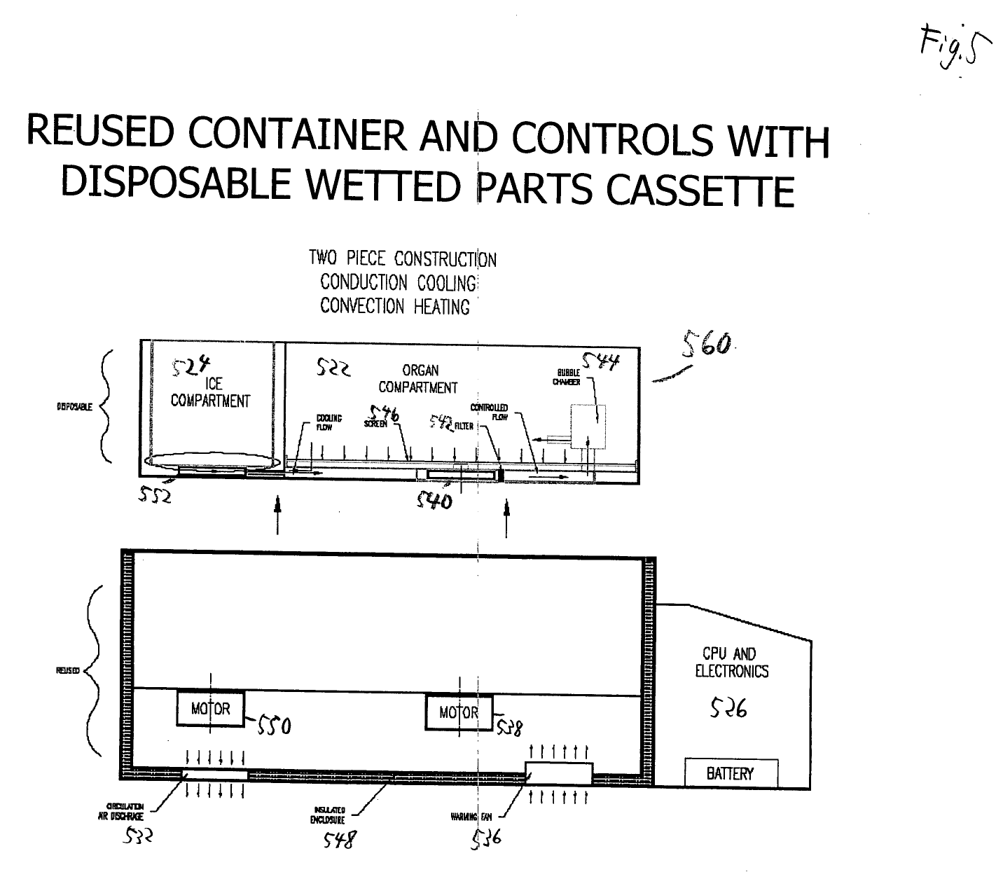

In another embodiment of the present invention 430, the temperature is regulated by conduction cooling and convection heating as shown in the schematic of FIG. 4. Perfusate circulation in this system is similar to that described in the convection model of FIG. 3. Perfusion fluid is circulated by a pump comprising a motor 438 and an impeller 440 controlled by the system controls 426 which pumps the fluid in a controlled manner through a filter 442, a bubble chamber 444 and finally through the tubing that connects the bubble chamber with the cannulated tissue into the tissue. After passing through the tissue, effluent passes through a screen 446 in the floor of the tissue container 422 and is pulled by a second pump comprising a second motor 450 and a second impeller 452 to flow past the removable ice container 424. The perfusion fluid is then cooled by conduction and pumped back into the tissue container 422. Warming is still accomplished by convection, as warm air is brought into the system by the warming fan 436 and circulated out the discharge vent 432. In one embodiment of the invention all of the components except for the system control computer 426 are disposable, as denoted in FIG. 4. In another embodiment of the invention, the parts of the apparatus that come in contact with the tissue, ice or perfusion fluid are contained in a disposable cassette 560, as shown in FIG. 5. In the embodiment diagrammed in FIG. 5, all of the components perform the same function as described in FIG. 4 and are numbered as such with an increment of 100.

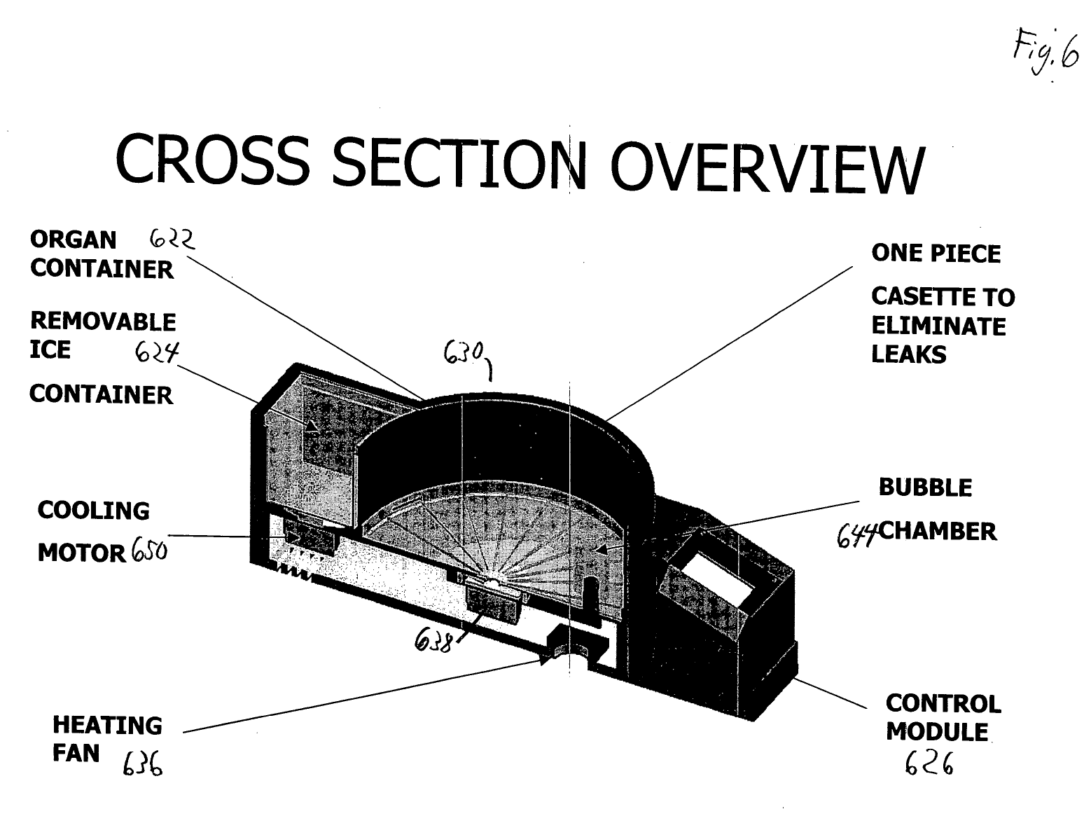

FIG. 6 shows a cross-sectional of the embodiment of the portable machine perfusion apparatus shown in general in FIG. 2 and diagrammed by the schematic of FIG. 4. All of the components shown perform the same function as described in FIG. 4 and are numbered as such with an increment of 100.

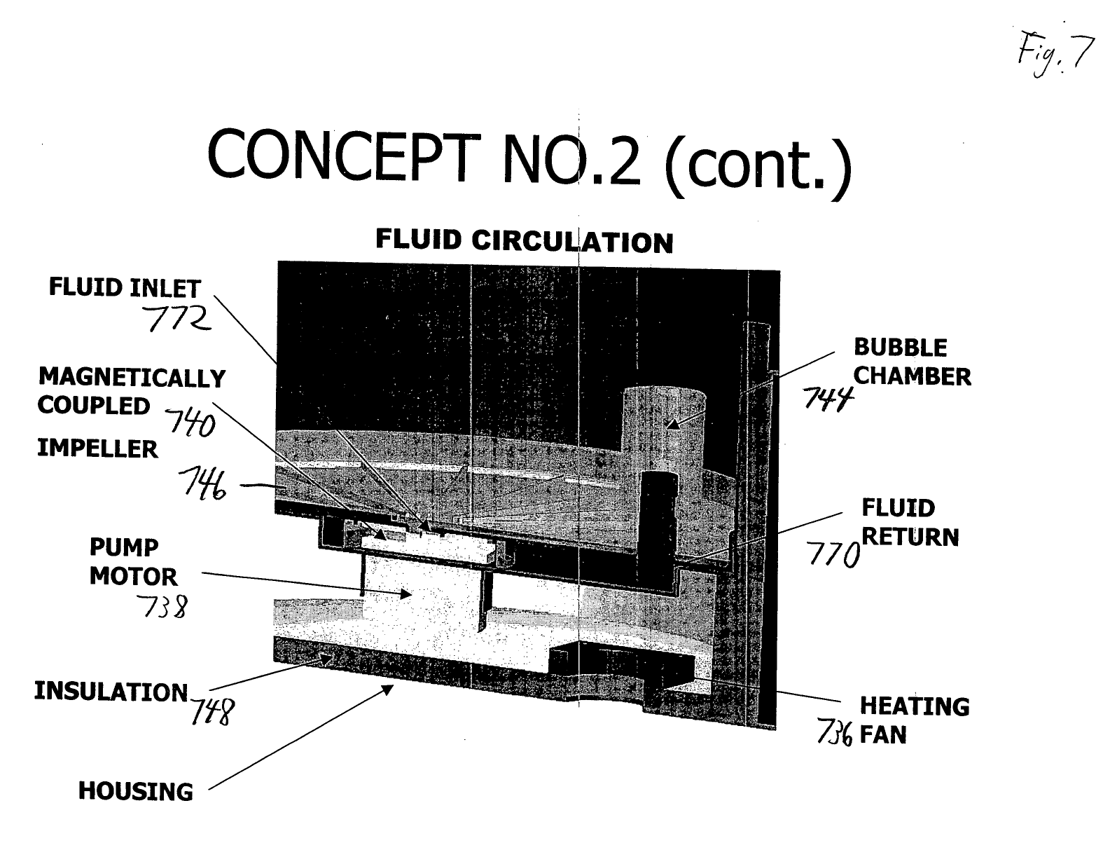

FIG. 7 is a close-up cross sectional schematic of an embodiment of the tissue container showing the fluid circulation system. The pump motor 738 drives the impeller 740 to pump perfusate through the bubble chamber 744 and into the tissue as described in FIGS. 3 and 4. After passing through the tissue, the effluent passes through a screen 746 on the floor of the tissue container and is collected in the fluid return 770 and pulled into the pump impeller 740 through the fluid inlet 772.

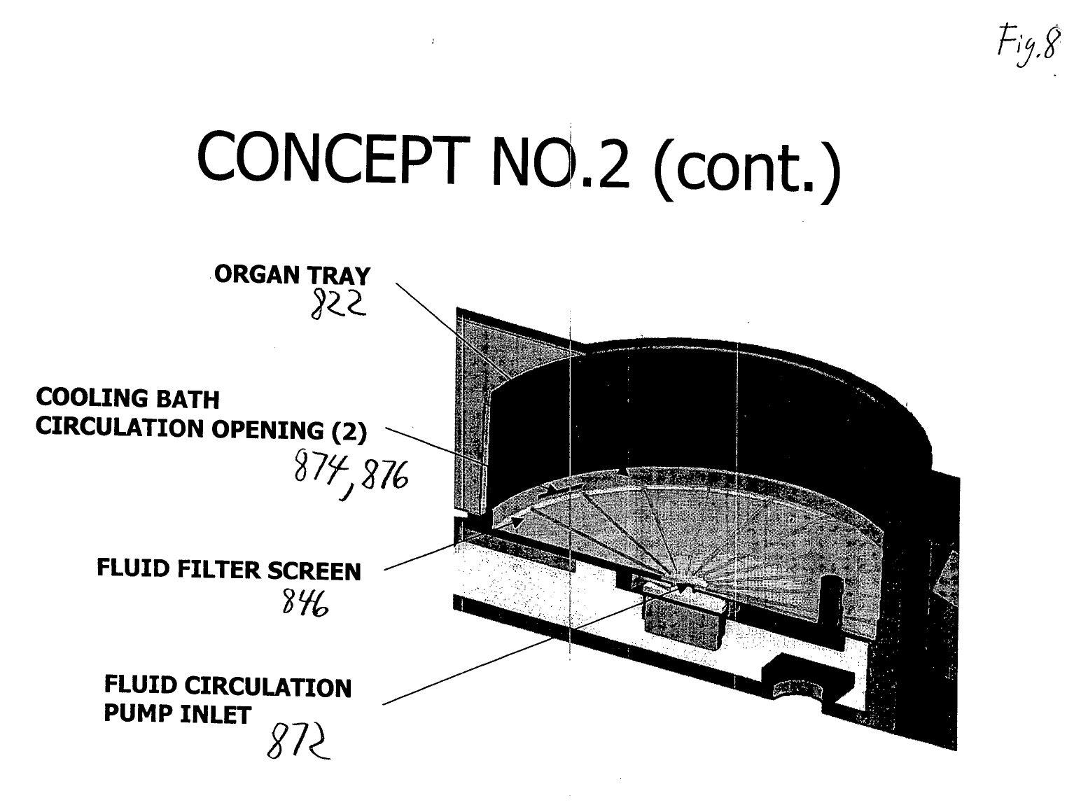

A more general cross-sectional schematic of a preferred embodiment of the tissue container for the apparatus diagrammed in FIG. 4 is shown in FIG. 8. After passing through the tissue as described in FIG. 7, the effluent from the tissue is re-circulated by passing through a screen 846 and being pulled through the pump fluid inlet 872. The perfusion fluid surrounding the tissue is circulated for conduction cooling through the impeller 452 as described in FIG. 4 through the circulation openings 874 and 876.

With reference to the pump to be used, in a preferred embodiment of the invention, the pump to be used is a centrifugal pump. In a more preferred embodiment the pump is a centrifugal pump that allows for delivery of a constant flow of perfusate with low-shear, laminar flow. Low-shear, laminar flow is preferred, as it allows the tissue to be perfused with little to no damage to the vascular tissues, in contrast to currently available kidney preservation apparatus. This type of flow also reduces intravascular turbulence, which can lead to perfusion damage. A non-limiting example of a pump for the preferred embodiment of the invention is the Bio-Pump® Plus centrifugal pump from Medtronic, Inc. (http://www.medtronic.com/cardsurgery/arrested_heart/centrifugal_pump.html). The Bio-Pump Plus has a vertical cutwater outlet design that reduces shear forces 40%. It will be apparent to those of skill in the art that various centrifugal pumps with various impeller designs delivering low-shear, laminar flow fall within the scope of the present invention.

In a preferred embodiment of the invention, the portable machine perfusion apparatus is powered by a battery power supply. An example of a preferred battery is a rechargeable sealed lead acid type battery. The sealed lead acid battery is safe to handle, has a long shelf life, and a deep duty cycle. It is easy to recharge, has a high charge density and a high cycle life. In a preferred embodiment of the invention, the machine perfusion apparatus contains at least two battery slots for performing a battery hot swap. As one of the objects of the present invention is a portable machine perfusion apparatus, the weight of the overall apparatus is to be kept to a minimum. By allowing the batteries to be exchanged, smaller and lighter weight batteries may still be used while still allowing the system to be operative for long periods of time, such as for hours or days.

In a preferred embodiment of the invention, the pump is controlled by the system controls using pulse width modulation control. Pulse-width modulation control works by switching the power supplied to the motor on and off very rapidly. The DC voltage is converted to a square-wave signal, alternating between fully on (nearly 12 v) and zero, giving the motor a series of power pulses. If the switching speed of such a system is high enough, the motor runs at a steady speed due to the momentum of its flywheel. The motor speed of a pulse-width modulation system can be varied by adjusting the duty cycle of the system. This type of control is advantageous for use in the present invention, because the output transistor is either on or off, not partly on as with normal regulation, so less power is wasted as heat and smaller heat-sinks can be used. The use of smaller heat sinks allows for the construction of a portable perfusion apparatus while still allowing critical temperature regulation.

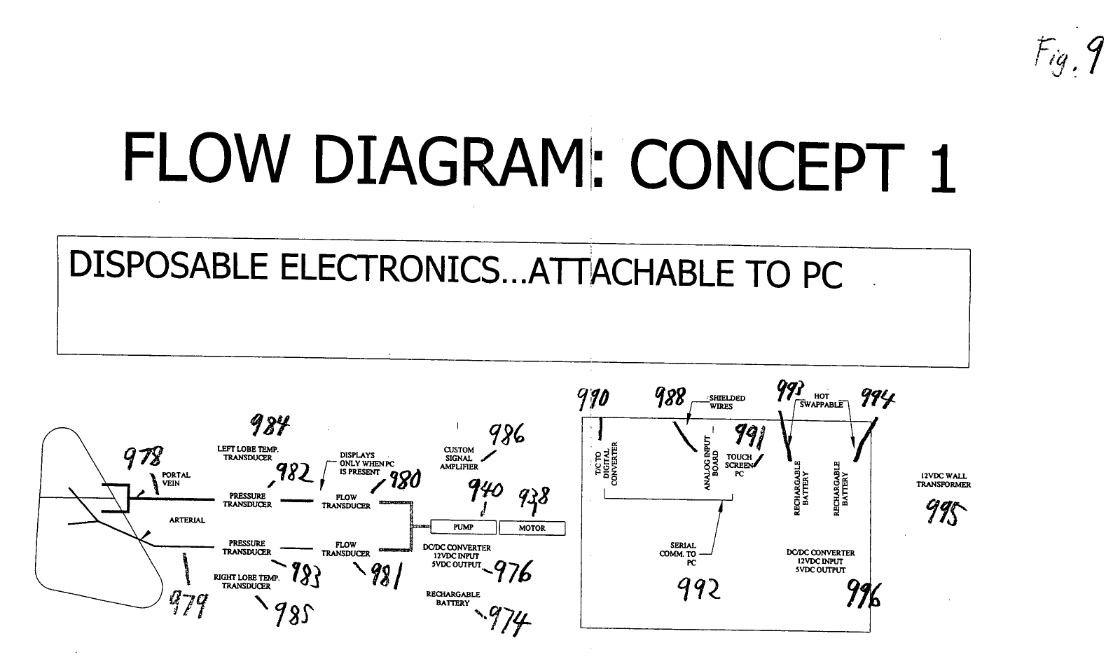

In one embodiment of the portable machine perfusion apparatus, the electronics of the system are disposable and are connected to reusable system controls or an outside computer. A flow diagram of this embodiment is shown in FIG. 9, using liver perfusion as a non-limiting example. A rechargeable battery 974 sends current through a 12V to 5V DC/DC converter 976 to drive a motor 938 that turns the pump impeller 940 to pump fluid through tubing for the left 978 (portal vein) and right 979 (hepatic artery) lobes. In each tube, perfusate flow passes through a flow transducer 980, 981 and a pressure transducer 982, 983 while the temperature of the perfusate is measured by a temperature transducer 984, 985. Signals from the flow and pressure transducers 980, 981, 982, 983 are amplified with a custom signal amplifier 986 and received by the analog input board 988. Signal from the temperature transducers 984, 985 are received by the T/C to digital converter 990. All received signals are communicated to a touch screen computer 991 through a serial communications component 992. The computer 991 is powered by either hot swappable rechargeable batteries 993, 994 or through house current flowing through a 12V DC wall transformer 995. Voltage is converted by a second 12V to 5V DC converter 996.

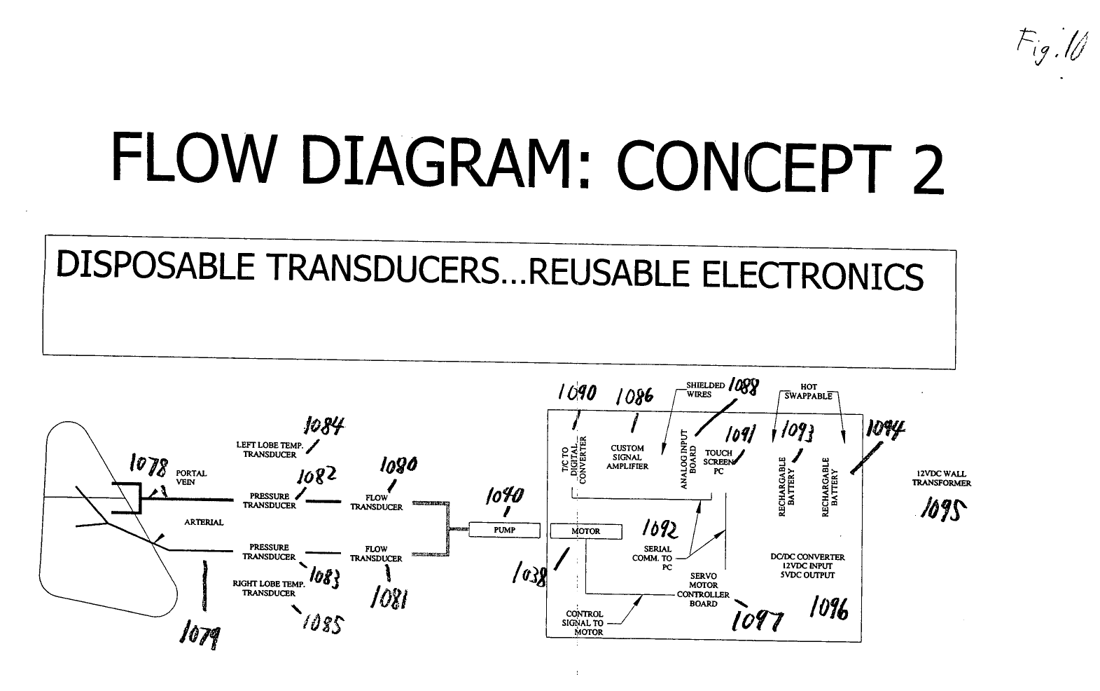

In another embodiment of the invention, the electronics of the portable machine perfusion apparatus are reusable, and only the transducer part of the system is disposable. A flow diagram of this embodiment is shown in FIG. 10, using liver perfusion as a non-limiting example. Components have the same function as in FIG. 9, and are numbered as such in an increment of 100. In the embodiment of FIG. 10, the motor 1038 is not disposable and is driven by the power source of the non-disposable components. In this embodiment, the speed of the motor is controlled by a servo motor controller board 1097, which receives input from the computer 1091.

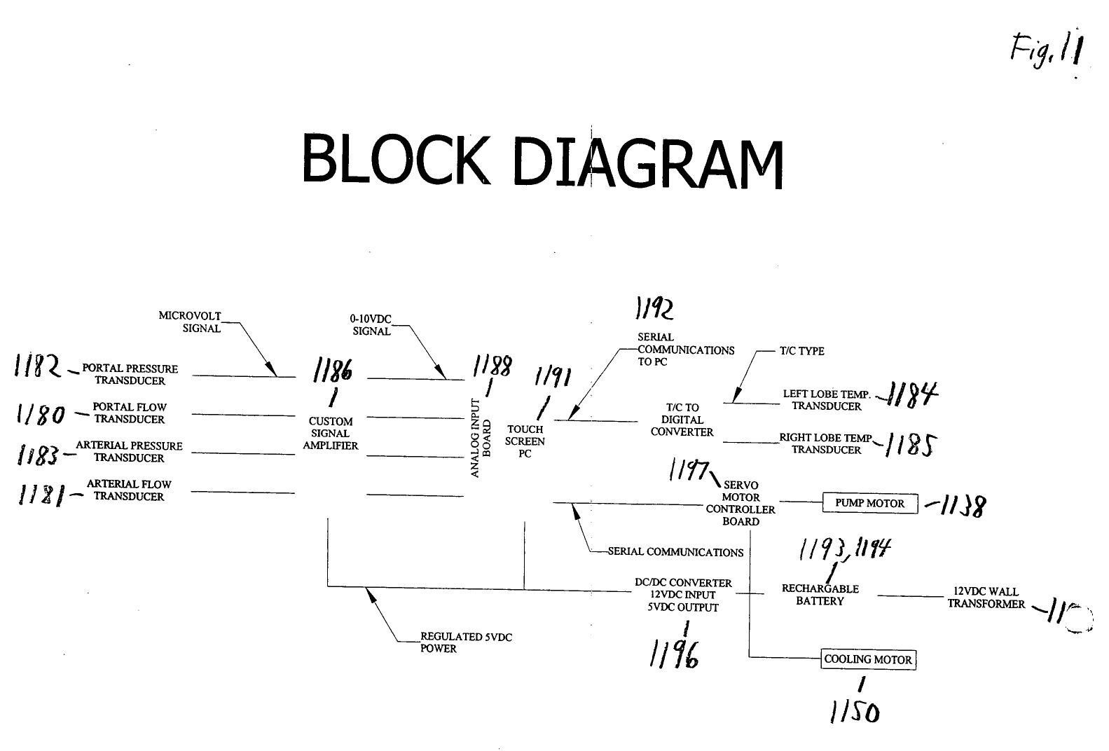

FIG. 11 is a block diagram further describing the control system diagrammed in FIG. 10. All components are numbered in the same manner, in an increment of 100. In FIG. 11, the cooling motor 1150 is also controlled by the servo motor controller board 1197.

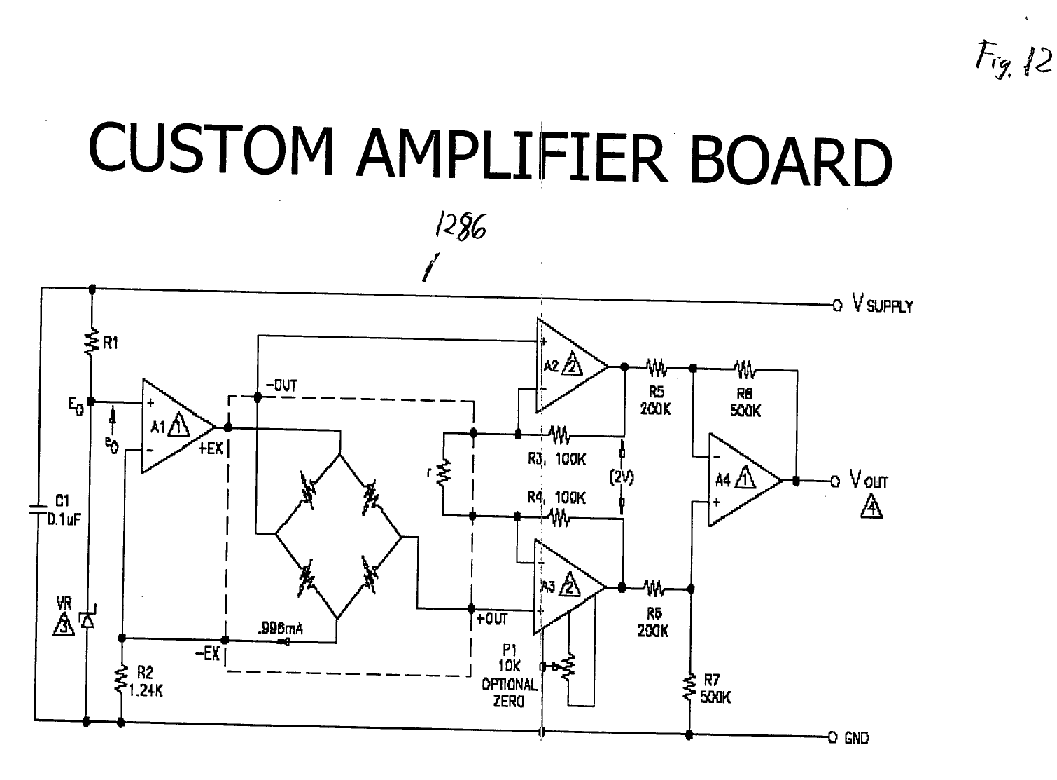

A preferred embodiment of an amplifier board to for the custom signal amplifier 986, 1086, 1186 of FIGS. 9, 10 and 11 is shown as a schematic drawing in FIG. 12.

Effective preservation of the tissue can be obtained at a variety of perfusate temperatures, including a range from hypothermic temperatures (about −10° C.) to standard human body temperature (about 37° C.). In a preferred embodiment of the invention, the temperature of the perfusate is maintained between 0° C. and 4° C. Likewise, the flow rate of the perfusate can be effective over a broad range of rates, from about 0.5 cubic centimeters per minute (cc/min) to about 5,000 cc/min.

With reference to the procurement of an organ, all cadaver donors must meet the standard criteria for brain death and procurement, and can be coordinated through an organ procurement organization. The donor liver could be procured by any common technique in the art. In a preferred embodiment, the surgical technique used for donor hepatectomy is a rapid en bloc procurement essentially as originally described by Starzl (Ann Surg 1989; 210:374-386). As described above, a segment of donor aorta is procured en bloc with the graft, which will not interfere with other organs being procured. Similarly, extra length of superior mesenteric/portal vein will be procured if the pancreas and small bowel are not being harvested. A core biopsy (tru cut) of the liver graft is performed. The biopsy is not interpreted at the time of procurement (frozen sections will not be performed) unless requested by the surgeon for clinical reasons, such as the appearance of the liver, unexpected findings, etc.

A summary of one possible surgical technique for procurement of a liver that may be used with the methods of the present invention is outlined below:

Surgical Technique of Rapid En bloc Procurement of liver grafts:
1. Long midline laparotomy and median sternotomy.
2. Exploration of the chest and abdomen to exclude malignancy.
3. Mobilization of the liver along the cardinal ligaments.
4. Kattell maneuver to expose the retroperitoneum.
5. Exposure of the distal aorta.
6. Exploration of the lesser sac and potential replaced left hepatic artery
7. Exploration of the Foramen of Winslow and indentification of a replaced right artery.
8. Exposure of the supraceliac aorta.
9. Exposure and cannulation of the inferior mesenteric vein.
10. Exposure of the common bile duct and irrigation of the biliary tract.
11. The left gastric and splenic artery are identified and ligated (this may be performed after crossclamp at the discretion of the procurement surgeon).
12. Mobilization of the pancreas for en bloc procurement with the liver, if the pancreas is to be utilized.
13. Heparinization of the donor.
14. Cannulation of the distal aorta.
15. Crossclamp, venting of the IVC, initiation of in situ flush with preservation solution, and topical cooling of the abdomen with ice slush (see protocol below).
16. Post flush en bloc removal of the liver and vascular pedical: includes division of the IVC, diaphragm surrounding the IVC, and dissection of the hepatic artery proximally to include the celiac artery. The donor aorta will be removed en bloc with the liver from the diaphragm proximally to the superior mesenteric artery distally.
17. The liver is weighed. The liver is again flushed on the back table with additional solution until the effluent is clear, and then packaged in a sterile container according to standard UNOS protocols for storage and transport.

The organ is flushed and prepared for preservation as in the non-limiting example that follows. It should be apparent to one of skill in the art that any potential protocol that prepares the organ for preservation could be used within the scope of the present invention, regardless of preservation solutions or volumes of solution used. In one protocol, the organ may be flushed with an organ preservation solution, such as University of Wisconsin (UW) solution, which is sold by Barr Laboratories as ViaSpan® and is described in U.S. Pat. Nos. 4,798,824 and 4,879,283. Alternatively, the organ may be flushed with the preservation solution Vasosol, as described in U.S. Patent Application publication 2002/0064768.

Summary of Liver preservation protocol.
1. Rapid en bloc multi organ surgical procurement as described by Starzl et al. (Ann Surg 1989; 210:374-386)
2. In situ aortic flush: 3 Liters of UW solution.
3. In situ portal flush: 1 Liter of UW solution.
4. Back table Hepatic artery flush: 300 cc of UW solution.
5. Back table Portal Flush: 650 cc of UW solution.
6. Bile duct: 50 cc of UW solution.
7. Packaged in 500 cc of UW solution.
8. Triple packaged in sterile bags in a UNOS approved cold storage container, packed with ice.
9. Livers are returned to the Preservation Unit for Machine Perfusion.

In an embodiment of the invention, the method of perfusion of the organ or other tissue occurs according to the following protocol. It is to be understood that variations of the protocol below will be recognized by one of skill in the art as falling within the scope of the present invention.

Protocol for Cannulation and Bench Work of Donor Liver or Machine Perfusion

All work on the donor liver will be performed in a class 100 sterile operating room, which is equipped with a laminar flow ventilation system.

Cannulation of the graft portal vein and hepatic artery is indirect and therefore atraumatic, preserving the graft vessels for anastomosis in the recipient. The machine perfusion protocol for procurement and cannulation were designed to perfuse the organ using segments of vessels that are far away from the vascular anastomoses performed in the recipient procedure. These procedures are performed by the donor surgeon with the assistance of the preservationist.

Preparation of the Inferior Vena Cava (IVC)

The IVC is dissected and cleaned. Small short hepatic veins are doubly ligated and divided. Phrenic branches are ligated and the diaphragmatic cuff is dissected off the bare area of the right lobe and discarded.

Preparation and Cannulation of the Aortic Conduit

The aortic segment is cleared and small lumbar branches are ligated with fine silk ties. The celiac axis is dissected to the common hepatic artery. The splenic and laft gastric arteries are tied near their origin. The hepatic artery is followed to the proper hepatic artery. The gastroduodenal artery is ligated with a 3-0 silk tie. Small phrenic and lymphatic branches are ligtated as necessary. An appropriate sized stainless steel reusable sterile cannula (Waters Medical, Rochester, Minn.) is introduced into the proximal segment of aorta and secured with umbilical tape. The distal segment of aorta is closed with a bulldog clamp. If there is a small distance of aorta below the celiac axis then the distal aorta is oversown in a running fashion with a 5-0 prolene suture.

Preparation and Cannulation of the Portal Conduit

The superior mesenteric vein and portal vein are dissected. Small branches are ligated with silk ties. A 22 gauge angiocath is inserted into the splenic vein orifice and threaded to the portal vein and secured with a silk tie. An appropriate sized stainless steel reusable sterile cannula (Waters Medical, Rochester, Minn.) is introduced into the proximal segment of the superior mesenteric vein and secured with a 0 silk tie.

Initiation of Perfusion

The liver is placed in a surgical basin and the aortic and portal cannulae are attached to the inflow lines on the machine perfusion apparatus via quick fix connectors. A temperature probe is placed into each lobe (segment 2 and 8). Twenty-two gauge angiocatheters are introduced into the proximal aortic conduit and into the mesenteric vein for pressure monitoring. The aortic and portal pressure catheters are connected to the machine perfusion device, which has a built in pressure transducer.

Perfusion is initiated at a flow rate of 0.66 cc/g/minute. The first 500 cc of effluent is collected and discarded, in order to remove residual cold storage solution within the graft. Priming of the circuit with excess machine preservation solution allows the effluent to pool in the basin and accomplish topical surface cooling of the liver in addition to the core cooling accomplished by vascular perfusion. The flow rate will be increased over the next 15 minutes to the target flow rate (see Table 1) based on the size of the graft. Flow rates will be adjusted lower if portal vein pressures rise above 8 mm Hg.

TABLE 1
Graft Weight Based Flow Rates for Liver Perfusion

| Liver Weight (g) | Flow range (cc/g/min) | Target flow (cc/minute) |
|---|---|---|
| 1100-1249 | 0.64-0.72 | 800 |
| 1250-1399 | 0.62-0.72 | 900 |
| 1400-1549 | 0.65-.71 | 1000 |
| 1550-1700 | 0.64-0.71 | 1100 |
| >1700 | 0.7 | 1200+ |

The target flow rate will be calculated in advance based on the size of the graft (0.6-0.7 cc/g/min) and the pump speed (RPM) will be adjusted to achieve target flow rates rather than a specific pressure. Trends in graft pressures will be noted and flow rate will be adjusted lower if portal pressure exceeds 8 mm Hg, to avoid any pressure injury to the hepatic sinusoidal endothelium. In preclinical work with human discard livers, arterial pressures at target flow ranges were never more than 30 mm Hg, so perfusion trauma to the hepatic arterial system is unlikely.

Changing of the Preservation Solution

The preservation solution is changed about every four hours accordingly. The four hour limit is intended to safeguard the graft and ensure maximal preservation. In discard liver studies there was no change in perfusate characteristics during four hours of observation. For comparison, in other methods of clinical kidney perfusion, the perfusate solution is never changed during the preservation period, which can be up to 24 hours duration in some instances. However, given the fact that this is a novel technique and due to the larger physical size of the liver, a protocol has been constructed which provides fresh perfusate every four hours. This time frame is designed to ensure maximal preservation characteristics of the perfusate. While longer intervals between perfusate changes may also be practical, longer time periods have not currently been tested.

Assessment of Perfusion and Graft Quality

Thirty minute assessments of the liver during MP will be performed, including both quantitative and qualitative variables:

Temperature

Flow

Portal pressure

Arterial pressure

Biochemistry: sodium, potassium, bicarbonate, calcium, glucose, ionized calcium

Osmolarity

Lactate and pH

Hourly samples will also be saved for later aspartate aminotransferase (AST), alanine aminotransferase (ALT) and lactate dehydrogenase (LDH) analysis, as are well known in the art. Graft biopsies, such as needle or wedge biopsies, may be taken for analysis both pre- and post-perfusion. Samples could be analyzed for changes in gene expression and using other common medical laboratory analysis.

After perfusion is completed, the graft will be packaged in a UNOS approved cold storage container for transportation to the operating room.

As concerns the preservation fluid to be used for perfusion of the tissue, in a preferred embodiment, the preservation fluid used is Vasosol, a novel preservation solution described in U.S. Patent Application Publication 2002/0064789. It has been shown that HMP with Vasosol improves early graft function in renal transplantation. (Guarrera et al. Transplantation. 2004 Apr. 27; 77(8):1264-8). It should be apparent to those of skill in the art that other preservation solutions can be used within the scope of the present invention, such as UW (Belzer) solution and the like.

In a preferred embodiment, the invention is used for ex vivo treatment and preservation of a tissue for transplantation. As described above, the tissue can be perfused using the apparatus of the present invention in a manner that provides metabolic substrates and removes waste products. During perfusion, the effluent of the tissue is periodically tested for various indicators of tissue health. If a tissue were to be deemed suitable for transplant by such tests, it could then be transplanted into the recipient. Further, selected therapeutic agents can be added to the perfusate. Such therapeutic agents would have the effect of improving the condition of the tissue or improving its ability to function in the recipient. The present invention can be used not only for preserving tissues for human transplants, but also has utility for preserving tissues for veterinary transplantation procedures of companion animals, livestock, and other living things for which transplantations are performed.

In another embodiment, the present invention can be used for a method of splitting tissues to achieve multiple grafts for transplant from one donor tissue. A liver, for example, may be split according to the following protocol. It is to be understood that variations of the protocol below will be recognized by one of skill in the art as falling within the scope of the present invention.

Protocol for Liver Splitting During Machine Perfusion

The liver is procured and perfusion is begun according to the protocols above. The hilar structures to the right and left lobe are dissected and identified. The graft parenchyma is scored sharply with a metzenbaum scissors and continued with blunt dissection in the desired plane of division. Crossing vessels that are encountered are clipped or ligated depending on size and surgeon preference. Hilar structures are left intact so that both graft segments may continue to undergo machine perfusion. Hilar structures are separated to complete the split at the termination of machine perfusion preservation. Two potential techniques of splitting the liver into two grafts include:

The whole liver may be split into a Right lobe graft (segments 1, 5, 6, 7, 8) for an adult and a Left lobe graft (segments 2, 3, 4) for a second small adult or large child.

The whole liver may be split into an Extended Right lobe for an adult (including segment 4) and a Left lateral segment graft (segment 2 and 3) for a child. A variation is the discard of segment 4 with a resulting right lobe and a left lateral graft remaining.

Other methods of liver splitting described in the art may be performed during machine perfusion within the scope of the present invention (see Noujaim et al. Am J Transplant 3:318-323 (2003), Yan et al. World J. Gastroenterol 11:4220-4224 (2005), Malago et al. World J. Surg. 26:275-282 (2002), and Renz et al. Ann Surg 239: 172-181 (2004) for non-limiting examples).

Liver splitting during machine perfusion has several advantages over currently performed liver splitting methods, which include ex situ (back table) splitting and in situ splitting during the procurement of the organ. Because the liver is being constantly perfused, it can be manipulated without concern of the graft being warmed by operative lights and the surgeon's hands because it is being cooled through the cold perfusion solution in the vasculature. Warming of the liver graft during splitting is thought to increase anaerobic metabolism and free radical injury. If the liver is split during machine perfusion, the core temperature of each segment of the liver does not change significantly.

Another advantage to liver splitting during machine perfusion is that the vessels of the organ are distended with perfusion solution, making them easy to clip and ligate. Additionally, because perfusion fluid is flowing through the vessels, the surgeon may be able to detect and repair leaking vessels before the organ is transplanted. This improves the hemostasis of the organ, and should allow for better graft function and stability. Liver splitting during machine perfusion is rapid and can be done under controlled flow conditions away from the donor site. Also, as it is performed ex situ, there is no risk to other tissues that might need to be harvested from the same donor.

In another embodiment, the present invention can be used for ex vivo treatment of a tissue to be re-implanted into the donor. Using such a method, selected therapeutic agents may be added to the perfusate to treat a disorder of the tissue or to improve tissue function once re-implanted.

In yet another embodiment, the present invention is used for pharmacological testing models. As a non-limiting example, a therapeutic agent could be tested in an isolated tissue, whereby the therapeutic agent is delivered and perfused into the tissue in blood, a blood-replacement or another perfusion solution. The effects of the therapeutic agent on the tissue can then be monitored over time.

It should be apparent that there are other embodiments of the invention that fall within the scope and spirit of the claims set forth below, and that the examples and variations provided herein are solely to help define specific embodiments of the invention.

### EXAMPLES

### Example 1

### Human Discard Protocol

Between May 2001 and March 2002, 10 non-transplantable human livers were obtained in accordance with the local Organ Procurement Organization. A model of atraumatic, centrifugal hypothermic machine perfusion (HMP) of the portal vein (PV) and hepatic artery (HA) was designed. During procurement, excess length of donor aorta and superior mesenteric/portal vein were procured with the graft; this allowed cannulation far from the area of the recipient anastomoses. Standard bench preparation of the graft, cannulation, and perfusion were performed in a class 100 sterile room.

Livers were hypothermically perfused with Vasosol solution for 5 to 10 h using the apparatus depicted in FIG. 1. The technique involved a flow-controlled system with target flow including temperature, flow, and HA and PV pressure were recorded every 30 minutes. Perfusate electrolytes were measured using an AVL automated blood gas analyzer.

Mean HMP time was 6.7±1.8 hours. Target flow was 0.7 mL/g liver/mg. PV and HA pressure ranged from 3 to 5 and 12 to 18 mm Hg, respectively. All grafts maintained adequate homogenous hypothermia (3° C. to 6° C.) during HMP. This was verified by serially measuring deep and surface temperatures of each liver segment. There were no technical or equipment failures that required termination of HMP. Effluent AST was measured in the last three discard livers. The values correlated strongly with the liver quality and the cold ischemia time at the initiation of HMP.

### Example 2

Animal Protocol

For proof of concept, a large animal liver transplant model was used. The study was conducted in accordance to the principles of laboratory animal care (NIH Publication No. 85-23, revised 1985).

Six miniature swine (24 to 32 kg) were used as donors. Standard liver procurement with in situ aortic flush with UW solution was performed. Donor swine were randomized to 12 hours of CS preservation (n=3) in ViaSpan (UW solution, Barr Laboratories, Inc. Pomona, N.Y., USA) or 12 hours of HMP using the apparatus depicted in FIG. 1 (n=3) with Vasosol solution.

After the preservation period, donor livers were transplanted orthotopically into six swine (26 to 31 kg) without venovenous bypass using the method described by Oike (Oike et al., Transplantation, 71:328, 2001). Recipient swine received intravenous dextrose infusion for 48 hours post-transplant. Animals also received oral tacrolimus and amoxicillin PO. Serum aspartate aminotrasferase and total bilirubin were measured to asses preservation injury. Surviving animals were sacrificed and necropsied on postoperative day 5.

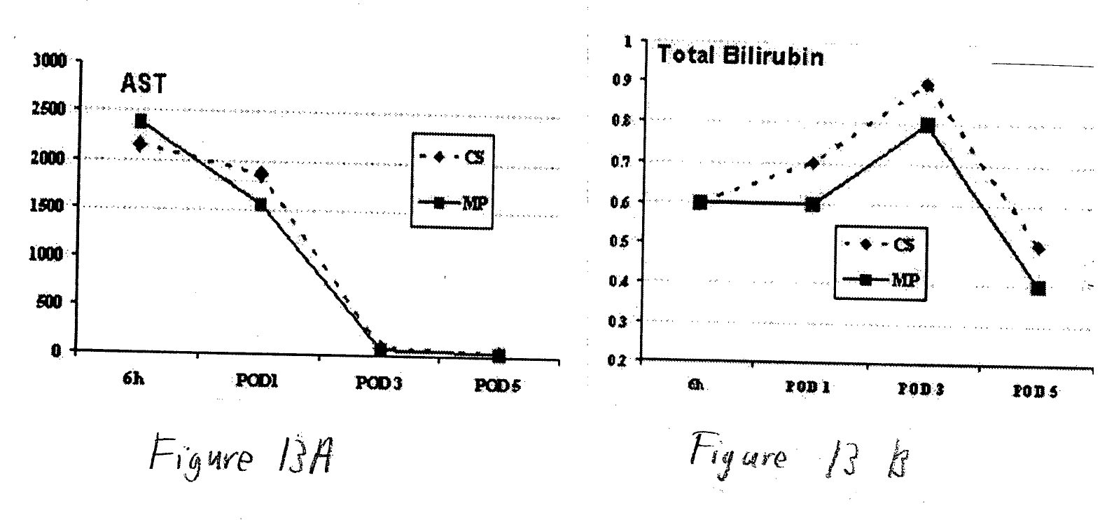

All recipient swine survived the liver transplantation procedure and awoke from anethesia. All swine had good initial liver function posttransplant and survived to postoperative day 5. Both groups had similar normalization of serum AST (FIG. 13A) and bilirubin (FIG. 13B) when monitored post-transplant. At necropsy, there was one arterial thrombosis in the CS group. There were no other preservation-related complications noted at the time of sacrifice.

The foregoing embodiments and advantages are merely exemplary and are not to be construed as limiting the present invention. The present teachings can be readily applied to other types of apparatuses. Also, the description of the embodiments of the present invention is intended to be illustrative and not to limit the scope of the claims. Many alternatives, modifications, and variations will be apparent to those skilled in the art.

I claim:

1. A perfusion apparatus for exsanguination or replacement of blood in a tissue, comprising:
a compartment for holding tissue to be treated; and
a centrifugal pump for delivering an aqueous medium to said tissue.

2. The apparatus of claim 1, wherein the output flow rate of the medium is from about 0.5 cubic centimeters per minute (cc/min) to about 5,000 cc/min.

3. The apparatus of claim 1, wherein the tissue is vascularized organ tissue.

4. The apparatus of claim 1, wherein the tissue is vascular tissue.

5. The apparatus of claim 1, wherein the apparatus is used to preserve an organ for transplantation.

6. The apparatus of claim 1, wherein the apparatus is used to preserve functional cells.

7. The apparatus of claim 1, wherein the apparatus is used for ex vivo treatment of an organ.

8. The apparatus of claim 1, further comprising a system for maintaining the temperature of the aqueous medium.

9. The apparatus of claim 1, wherein the aqueous medium is maintained at a temperature from about −10° C. to about 37° C.

10. A portable perfusion apparatus for exsanguination or replacement of blood in a tissue, comprising:
a compartment for holding tissue to be treated; and
a centrifugal pump for delivering an aqueous medium to said tissue.

11. The apparatus of claim 10, wherein the output flow rate of the medium is from about 0.5 cubic centimeters per minute (cc/min) to about 5,000 cc/min.

12. The apparatus of claim 10, wherein the tissue is vascularized organ tissue.

13. The apparatus of claim 10, wherein the tissue is vascular tissue.

14. The apparatus of claim 10, wherein the apparatus is used to preserve an organ for transplantation.

15. The apparatus of claim 10, wherein the apparatus is used to preserve functional cells.

16. The apparatus of claim 10, wherein the apparatus is used for ex vivo treatment of an organ.

17. The apparatus of claim 10, further comprising a system for maintaining the temperature of the aqueous medium.

18. The apparatus of claim 10, wherein the aqueous medium is maintained at a temperature from about −10° C. to about 37° C.

19. The apparatus of claim 10, further comprising a portable power source.

20. The apparatus of claim 19, wherein the portable power source comprises one or more batteries.

21. A method for exsanguination or replacement of blood in a tissue using a perfusion apparatus comprising the steps of:
(a) depositing in the perfusion apparatus a tissue in need of preservation or treatment;
(b) introducing into the perfusion apparatus an aqueous medium; and
(c) delivering the aqueous medium to said tissue through a centrifugal pump.

22. The method for exsanguination or replacement of blood in a tissue according to claim 21, wherein the output flow rate of the medium is from about 0.5 cubic centimeters per minute (cc/min) to about 5,000 cc/min.

23. The method for exsanguination or replacement of blood in a tissue according to claim 21, wherein the aqueous medium is a solution maintained at a temperature from about −10° C. to about 37° C.

24. The method for exsanguination or replacement of blood in a tissue according to claim 21, wherein the aqueous medium is used for preserving an organ for transplantation.

25. The method for exsanguination or replacement of blood in a tissue according to claim 21, wherein the aqueous medium is used for ex vivo treatment of the tissue.

26. The method for exsanguination or replacement of blood in a tissue according to claim 21, further comprising administering a therapeutic agent in combination with the aqueous medium.

27. The method for exsanguination or replacement of blood in a tissue according to claim 21, wherein the tissue is used for autologous re-implantation.

28. The method for exsanguination or replacement of blood in a tissue according to claim 21, wherein said method is used for pharmacological study.

29. The method for exsanguination or replacement of blood in a tissue according to claim 21, wherein the aqueous medium is Vasosol.

30. The method for exsanguination or replacement of blood in a tissue according to claim 21, wherein the aqueous medium is blood.

31. The method for exsanguination or replacement of blood in a tissue according to claim 21, wherein the aqueous medium is artificial blood.

32. The method for exsanguination or replacement of blood in a tissue according to claim 21, wherein the tissue is a liver.

33. The method for exsanguination or replacement of blood in a tissue according to claim 21, wherein the tissue is a heart.

34. The method for exsanguination or replacement of blood in a tissue according to claim 21, wherein the tissue is a kidney.

35. The method for exsanguination or replacement of blood in a tissue according to claim 21, wherein the tissue is a pancreas.

36. The method for exsanguination or replacement of blood in a tissue according to claim 21, wherein the tissue is a lung.

37. A method of preserving a tissue for a veterinary transplant using a perfusion apparatus comprising the steps of:
(a) receiving in the perfusion apparatus a tissue in need of preservation or treatment;
(b) introducing into the perfusion apparatus an aqueous medium.

38. A method of splitting a tissue using a perfusion apparatus comprising the steps of:
(a) receiving in the perfusion apparatus a tissue in need of preservation or treatment;
(b) introducing into the perfusion apparatus an aqueous medium
(c) splitting the tissue by a surgical method while perfusion of the tissue is ongoing.

39. The method of splitting a tissue using a perfusion apparatus according to claim 38, wherein the output flow rate of the medium is from about 0.5 cubic centimeters per minute (cc/min) to about 5,000 cc/min.

40. The method of splitting a tissue using a perfusion apparatus according to claim 38, wherein the aqueous medium is a solution maintained at a temperature from about −10° C. to about 37° C.

41. The method of splitting a tissue using a perfusion apparatus according to claim 38, wherein the aqueous medium is Vasosol.

42. The method of splitting a tissue using a perfusion apparatus according to claim 38, wherein the tissue is a liver.

43. The method of splitting a tissue using a perfusion apparatus according to claim 38, wherein the tissue is a heart.

44. The method of splitting a tissue using a perfusion apparatus according to claim 38, wherein the tissue is a kidney.

45. The method of splitting a tissue using a perfusion apparatus according to claim 38, wherein the tissue is a pancreas.

46. The method of splitting a tissue using a perfusion apparatus according to claim 38, wherein the tissue is a lung.
